(19) United States
(12) Patent Application Publication
(10) Pub. No.: US 2020/0229693 A1
(43) Pub. Date: Jul. 23, 2020

(54) MEASURING METHOD FOR DETERMINING AN ASTIGMATISM OF AN EYE

(71) Applicant: Carl Zeiss Meditec AG, Jena (DE)

(72) Inventor: Mathis Piper, Oberkochen (DE)

(21) Appl. No.: 16/751,192

(22) Filed: Jan. 23, 2020

(30) Foreign Application Priority Data
Jan. 23, 2019 (DE) 10 2019 101 618.5

(51) Int. Cl. A61B 3/103; A61B 3/00

(52) U.S. Cl. CPC A61B 3/1035; A61B 3/0008

(57) ABSTRACT

A measuring method for determining an astigmatism of an eye with a confocal refractometer providing a measurement beam path, in which an optical arrangement having an adaptive optical component with a variable cylinder power and a variable cylindrical axis position is arranged to compensate for an astigmatism of the eye in the wavefront of the measurement beam path. In a first measurement, the cylinder power of the adaptive optical component for a first fixed cylindrical axis position of the adaptive optical component is varied until the measured intensity becomes maximal. In a second measurement, the cylinder power of the adaptive optical component for a second fixed axis position—different than the first—of the adaptive optical component is varied until the measured intensity is maximal. The power and axis position of the astigmatism of the eye are determined from first and second measurement values.

### CROSS REFERENCE TO RELATED APPLICATIONS

This application claims priority to German patent application DE 10 2019 101 618.5, filed Jan. 23, 2019, the entire content of which is incorporated herein by reference.

### TECHNICAL FIELD

The disclosure relates to a measuring method for determining an astigmatism of an eye with the aid of a confocal refractometer providing a measurement beam path, in which is arranged an optical arrangement having an adaptive optical component having a variable cylinder power and a variable cylindrical axis position in order to compensate for an astigmatism of the eye in the wavefront of the measurement beam path.

### BACKGROUND

Such a measuring method is generally known from the subsequently published document DE 10 2017 117 925 A1. Said document also describes a confocal refractometer that can be used to carry out the measuring method. A further confocal refractometer is known from the document US 2015/0109580 A1.

Such a confocal refractometer can be used to measure the spherical equivalent SE of the ametropia of a patient's eye, an astigmatism of the patient's eye including the axis position of the astigmatism of the patient's eye. Both the spherical equivalent SE and the astigmatism C are usually specified in diopters (D). Typically, eyes with the spherical equivalent of SE<0 D are referred to as nearsighted or myopic, while eyes with SE>0 D are referred to as farsighted or hyperopic. Patients' eyes with SE≈0 D are referred to as having spherically perfect vision or as emmetropic. The astigmatism C specifies the difference between the refractive powers of the eye in two mutually perpendicular principal meridians. The axis position φ specifies the position of these principal meridians, represents an angle and is specified in the unit of degrees (°).

The following convention is used in the description of the present disclosure: The astigmatism C is always positive and satisfies C>0 D. In the two principal meridians, the defective vision of the patient's eye is described by SE±(1/2) C. The axis position φ describes the position of that principal meridian with the defective vision SE +(1/2) C. For known values SE, C, and φ, it is possible to produce a spectacle lens that corrects the defective vision of the patient's eye. It is also possible to use other conventions for describing defective vision, but they can always be converted into the convention indicated above.

In the context of the present disclosure, “confocal” is understood to mean an optical system that enables “point-to-point” imaging. A pinhole stop illuminated with measurement light by a light source of the refractometer is imaged onto the retina of the patient's eye as a measurement light beam, with the result that a light spot is produced on the retina. For this purpose, the measurement light beam is focused onto the retina by a focusing device of the optical arrangement of the refractometer, with the result that the light spot on the retina can be chosen to be as small as possible. Measurement light incident in the region of the light spot is partly scattered or reflected by the retina, with the result that light energy emerges from the eye as measurement light reflected back. A confocal stop is positioned in a plane conjugate to the retina, said stop at least partly transmitting the measurement light reflected back from the eye, wherein the intensity of the back-reflected measurement light transmitted by the stop is measured by a measuring module with a light detector. Instead of physical stops, it is also possible to use the end of an optical fiber as a confocal stop on the light source side and as a confocal stop on the light detector side.

In the case of the measuring method for determining an astigmatism of a patient's eye with the aid of a confocal refractometer as known from the document cited in the introduction, after the spherical equivalent of the ametropia of the patient's eye has been measured, firstly the axis position of the astigmatism of the patient's eye (with compensated spherical equivalent) is measured. For this purpose, either a known cylinder power or a small cylinder power is set at the adaptive optical component. Afterward, the axis position of the adaptive optical component is varied, and the intensity of the measurement light reflected back is determined. The sought axis position of the astigmatism is found at maximum intensity of the measurement light reflected back. In a further step, for the axis position determined, the cylinder power of the adaptive optical component is then varied until the intensity of the measurement light reflected back becomes maximal again, from which the power of the astigmatism can then be determined.

In the case of the known measuring method, the astigmatism is thus determined by firstly carrying out an axis measurement and then a cylinder measurement.

Measurements carried out on subjects in accordance with the known measuring method have shown that measurement signals that cannot be evaluated often occur during the axis measurement with the known measuring method. As a result, the power of the astigmatism and the axis position thereof cannot be determined reliably.

### SUMMARY

Therefore, it is an object of the disclosure to provide a measuring method for determining an astigmatism of an eye with the aid of a confocal refractometer with which the power of the astigmatism and the axis position thereof can be determined reliably.

According to a first aspect of the disclosure, the object is achieved by a measuring method for determining an astigmatism of an eye with the aid of a confocal refractometer providing a measurement beam path, in which is arranged an optical arrangement having an adaptive optical component having a variable cylinder power and a variable cylindrical axis position in order to compensate for an astigmatism of the eye in the wavefront of the measurement beam path, including the following steps: directing a measurement light beam onto the eye such that a light spot is produced on the retina of the eye, measuring an intensity of measurement light reflected back from the retina, wherein in a first measurement the cylinder power of the adaptive optical component for a first fixed cylindrical axis position of the adaptive optical component is varied until the measured intensity becomes maximal in order to obtain a first measurement value, and in a second measurement the cylinder power of the adaptive optical component for a second fixed axis position—different than the first—of the adaptive optical component is varied until the measured intensity is maximal in order to obtain a second measurement value, determining the power and axis position of the astigmatism of the eye at least from the first and second measurement values.

With the measuring method according to an aspect of the disclosure, the astigmatism is determined by carrying out a first and a second measurement, which in each case are also referred to as cylinder measurements in the present description. In the case of the measuring method for determining the astigmatism of a patient's eye, a measurement of the axis position of the astigmatism becomes superfluous. Rather, the axis position of the astigmatism of the patient's eye is determined computationally from the two cylinder measurements. Inaccurate measurement signals or measurement signals that cannot be evaluated are thus avoided. The measuring method in accordance with the first aspect of the disclosure is distinguished by its high robustness and accuracy.

In the case of the first cylinder measurement, a first fixed cylindrical axis position is set at the adaptive optical component. The first fixed cylindrical axis position is arbitrary, but known on account of the setting. For this first fixed cylindrical axis position, the cylinder power of the adaptive optical component is varied until the measured intensity becomes maximal. A first measurement value is yielded as the measurement result, which first measurement value is also referred to as cylinder measurement value in the present description. After the first cylinder measurement, a second cylinder measurement is carried out, in the case of which a second fixed axis position is set at the adaptive optical component, said second fixed axis position being different from the first axis position. For this second axis position of the adaptive optical component, the cylinder power of the adaptive optical component is varied until the measured intensity is maximal again. A second measurement value or cylinder measurement value is obtained as the result.

The first cylinder measurement value and the second cylinder measurement value are accordingly obtained by an intensity measurement of the measurement light reflected back. Both in the case of the first cylinder measurement and in the case of the second cylinder measurement, the cylinder power of the adaptive optical component is varied until the measured intensity of the measurement light reflected back is maximal. The two cylinder measurement values can be gleaned from the setting of the respective cylinder power of the adaptive optical component. The astigmatism and the axis position of the patient's eye are then determined by calculation from the first and second cylinder measurement values, and optionally the first axis position and the second axis position of the adaptive optical component. The first and second axis positions of the adaptive optical component, as already mentioned, are arbitrary, but known on account of the setting of the adaptive optical component.

Typically, the spherical equivalent of the patient's eye is measured before the first cylinder measurement. For this purpose, the optical arrangement of the confocal refractometer is furthermore designed to vary a focus position of the measurement beam path in order to compensate for a spherical equivalent of the ametropia of the eye in the wavefront of the measurement beam path, wherein before the first measurement (cylinder measurement) with the aid of the optical arrangement the focus position of the measurement beam path is varied until the measured intensity of the measurement light reflected back from the retina is maximal.

The advantage of this measure is that the two cylinder measurements are carried out with a compensated spherical equivalent of the ametropia, and the cylinder measurements are thus independent of the spherical equivalent.

The cylinder power of the adaptive optical component can be set to zero or neutral during the variation of the focus position. As a result, the spherical equivalent of the ametropia can be optimally compensated for by varying the focus position of the optical arrangement.

With further preference, the spherical equivalent of the ametropia of the eye is determined from the setting of the optical arrangement in which the spherical equivalent of the ametropia is compensated for.

In this configuration, the measuring method thus allows not only the measurement of the astigmatism, but also the measurement of the spherical equivalent of the ametropia of the patient's eye. In this configuration, the measuring method in accordance with the first aspect requires a total of just three measuring steps, specifically one measuring step for measuring the spherical equivalent of the ametropia and two measuring steps for measuring the astigmatism including the axis position thereof.

The first measurement value a and the second measurement value b can be linked with the power of the astigmatism C_{eye} of the eye and the axis position φ in a simple manner by: a=−C_{eye}·cos(2β₁), b=−C_{eye}·cos(2β₂), where β₁=φ−φ₁ and β₂=φ−φ₂, wherein φ₁ is the axis position of the adaptive optical component in the first measurement and φ₂ is the axis position of the adaptive optical component in the second measurement.

The axis position φ of the astigmatism of the eye can thus be determined in a simple manner by calculation from the first measurement value a, the second measurement value b, the first axis position φ₁ of the adaptive optical component and the angle Δη between the first axis position and the second axis position of the adaptive optical component in accordance with the following equation:

φ=(1/2)·arctan((a·cos(2Δβ)−b)/(a·sin(2Δβ)))+φ₁

Typically, the second axis position of the adaptive optical component (AOE) differs from the first axis position of the adaptive optical component (AOE) by an angle in the range of 30° to 45°.

The calculation of the power and the axis position of the astigmatism of the eye from the first and second measurement values is simplified even further if the first axis position of the adaptive optical component is set to 0° with respect to the horizontal axis of the eye (12) and the second axis position is altered by 45° relative to the first axis position, as is provided in one exemplary embodiment.

By means of the measure mentioned above, the power of the astigmatism C_{eye} of the eye can be determined just from the first measurement value a and the second measurement value b in accordance with the following equation:

C_{eye}=−(a²+b²)^{1/2}

The axis position φ of the astigmatism of the eye can likewise be determined in a simple manner just from the first measurement value (a) and the second measurement value (b) in accordance with the following equation:

φ=1/2 arctan (−b/a)

In this calculation method, the power of the astigmatism of the eye can be calculated independently of and before the determination of the axis position of the astigmatism of the eye, and vice versa.

According to a second aspect of the disclosure, the object is achieved by a measuring method for determining an astigmatism of an eye with the aid of a confocal refractometer providing a measurement beam path, in which is arranged an optical arrangement having an adaptive optical component having a variable cylinder power and a variable cylindrical axis position in order to compensate for an astigmatism of the eye in the wavefront of the measurement beam path, and wherein the optical arrangement is furthermore configured to vary a focus position of the measurement beam path to compensate for a spherical equivalent of the ametropia of the eye in the wavefront of the measurement beam path, the method including the following steps: directing a measurement light beam onto the eye such that a light spot is produced on the retina of the eye, measuring an intensity of measurement light reflected back from the retina, wherein in a first measurement the focus position of the measurement beam path for a fixed first cylinder power of the adaptive optical component and a fixed axis position of the adaptive optical component is varied until the measured intensity of the measurement light reflected back from the retina is maximal in order to obtain a first measurement value, and in a second measurement the focus position of the measurement beam path for the first cylinder power and a second axis position—different than the first—of the adaptive optical component is varied until the measured intensity of the measurement light reflected back from the retina is maximal in order to obtain a second measurement value, and determining the power of the astigmatism of the eye and the axis position thereof at least from the first and second measurement values.

In the case of the measuring method in accordance with the second aspect of the disclosure, an astigmatism of a patient's eye is determined by carrying out two measurements based in each case on an intensity measurement for measuring the spherical equivalent of the ametropia of the patient's eye. In the present description, the two measurements are also referred to as SE measurements, wherein SE stands for the spherical equivalent of the ametropia of the patient's eye.

As in the measuring method in accordance with the first aspect of the disclosure, the intensity of measurement light reflected back from the retina is measured in each case. In the first SE measurement, a fixed first cylinder power is set at the adaptive optical component, which cylinder power can in particular be not equal to 0 D. In this case, the first cylinder power is arbitrary and known on the basis of the setting of the adaptive optical arrangement. Furthermore, a fixed axis position of the cylinder of the adaptive optical component is set at the adaptive optical component, which axis position is correspondingly known and can be arbitrary. For a set first cylinder power and first axis position of the adaptive optical component, the focus position of the optical arrangement is varied until the measured intensity of the measurement light reflected back from the retina is maximal. A first measurement value is obtained as the result, said first measurement value also being referred to as the SE measurement value in the present description.

In the second SE measurement, a second fixed axis position different than the first axis position is set at the adaptive optical component. The first cylinder power of the adaptive optical arrangement set in the first SE measurement is maintained in this case. Afterward, the focus position of the optical arrangement is varied again until the measured intensity of the measurement light reflected back from the retina is maximal. A second SE measurement value is obtained as a result of the measurement. In the subsequent step, the power of the astigmatism of the eye and the axis position thereof can be determined from the first and second SE measurement values and optionally the set first cylinder power and axis position of the adaptive optical component.

An advantage of the measuring method in accordance with the second aspect is that the astigmatism of the patient's eye and the axis position thereof can be determined with a total of just two measuring steps.

In the case of a patient's eye afflicted with astigmatism, two intensity maxima usually occur during the variation of the focus position of the measurement beam path. Accordingly, in one configuration of the measuring method in accordance with the second aspect of the disclosure, in the first and second measurements the focus position is varied until the measured intensity of the measurement light reflected back in each measurement has respectively a first and a second intensity maximum, wherein the first and second measurement values are respectively determined from that setting of the optical arrangement which is associated with the first and second intensity maxima. If, e.g., the optical arrangement of the confocal refractometer for varying the focus position of the measurement beam path has one or more movable optical elements, the first and respectively the second SE measurement value can be obtained from the displacement distance of the optical element(s) between the two respective intensity maxima.

However, it may happen that the astigmatism of the patient's eye was randomly compensated for or almost compensated for as a result of the setting of the first cylinder power of the adaptive optical component, and so only one intensity maximum occurs during the variation of the focus position of the measurement beam path in the first measurement. In this case, if the first cylinder power of the adaptive optical component is altered, two well-separated intensity maxima can be detected.

As in the case of the measuring method in accordance with the first aspect of the disclosure, a simple algorithm is provided for the calculation of the power of the astigmatism of the patient's eye and the axis position of the astigmatism of the patient's eye. In this regard, the first measurement value A₁ and the second measurement value A₂ are linked with the power of the astigmatism C_{eye} of the eye by:

A_{1,2}=(C_{AOE}²+2C_{AOE}C_{eye}·cos(2β_{1,2})+C_{eye}²)^{1/2}

where C_{AOE} is the set first cylinder power of the adaptive optical component, β₁ is the angle between the principal meridians of the cylinders of the adaptive optical component and of the eye in the first measurement, and β₂ is the angle between the principal meridians of the cylinders of the adaptive optical component and of the eye in the second measurement.

The abovementioned relationship between the first and second measurement values A_{1,2} and the power of the astigmatism can be simplified further if the axis position of the adaptive optical component in the second SE measurement is rotated by 90° relative to the axis position of the adaptive optical component in the first SE measurement. The power of the astigmatism C_{eye} can then be determined in a simple manner from the first and second measurement values A_{1,2} and the set cylinder power C_{AOE} of the adaptive optical component:

C_{eye}=(A₁ ²−C_{AOE}²−1/2(A₁²−A₂²))^{1/2}.

Likewise, the angle β₁ between the principal meridians of the cylinders of the adaptive optical component and of the eye in the first measurement can be calculated in a simple manner by:

β₁=1/2 arccos((A₁²−A₂²)/(4C_{AOE} C_{eye})).

The axis position φ of the astigmatism of the eye then results from the relationship φ=φ₁+β₁, wherein φ1 is the axis position of the adaptive optical component in the first SE measurement.

The measuring method in accordance with the second aspect of the disclosure enables faster measurements of the astigmatism since only two measuring steps are required.

### BRIEF DESCRIPTION OF THE DRAWINGS

The disclosure will now be described with reference to the drawings wherein:

FIG. 1 shows a basic schematic diagram of a confocal refractometer according to a first exemplary embodiment of the disclosure;

FIG. 2 shows a basic schematic diagram of a confocal refractometer according to a second exemplary embodiment of the disclosure;

FIG. 3 shows an optical unit of a confocal refractometer in various settings of an optical arrangement for determining different instances of defective vision of patients' eyes according to an exemplary embodiment of the disclosure;

FIG. 4 shows a diagram of intensities—measured by way of example—of measurement light reflected back for various patients' eyes free of astigmatism and having a different spherical equivalent of the ametropia;

FIG. 5 shows a diagram of intensities—measured by way of example—of measurement light reflected back for various astigmatisms for one value of the spherical equivalent of the ametropia;

FIG. 6 shows a flowchart for elucidating a measuring method for determining an astigmatism of a patient's eye in accordance with an exemplary embodiment;

FIG. 7 shows a flowchart illustrating individual steps during a first cylinder measurement in the measuring method in accordance with FIG. 6;

FIG. 8 shows a flowchart illustrating individual steps of a second cylinder measurement of the measuring method in FIG. 6;

FIG. 9 schematically shows a front view of a patient's eye with a reference coordinate system;

FIG. 10 shows a flowchart for elucidating a measuring method for determining an astigmatism of a patient's eye in accordance with a further exemplary embodiment;

FIG. 11 shows a flowchart illustrating individual steps of a first SE measurement in the measuring method in accordance with FIG. 10; and

FIG. 12 shows a flowchart illustrating individual steps of a second SE measurement in the measuring method in accordance with FIG. 10.

### DESCRIPTION OF EXEMPLARY EMBODIMENTS

Further advantages and features are evident from the following description and the attached drawing. It goes without saying that the aforementioned features and those yet to be explained below can be used not only in the respectively specified combination but also in other combinations or on their own, without departing from the scope of the present disclosure.

Before one exemplary embodiment of a measuring method for determining an astigmatism of a patient's eye is described, firstly the construction of a confocal refractometer is described by way of example. It goes without saying that such a confocal refractometer can be integrated into an ophthalmic surgical microscope. For reasons of simpler illustration, however, an illustration of an integration of the refractometer into a surgical microscope is dispensed with here.

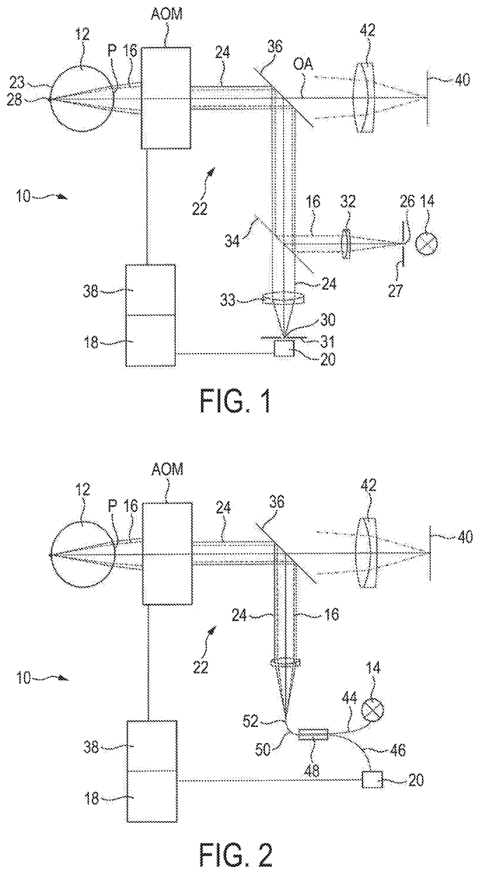

FIG. 1 shows a confocal refractometer 10. The confocal refractometer 10 can generally be used for determining the refraction of a patient's eye 12. The confocal refractometer 10 includes a measurement light source 14 for generating a measurement light beam 16. The measurement light beam is indicated by dashed lines in FIG. 1. The confocal refractometer 10 furthermore includes a measuring module 18 including a light detector 20 for measuring an intensity of reflected-back measurement light 24, which is indicated by solid lines in FIG. 1.

The refractometer 10 includes an optical unit 22, through which a measurement beam path passes, in order to direct the measurement light beam 16 onto the retina 23 of the eye 12 and to feed measurement light 24 reflected back at the retina 23 to the light detector 20. The measurement beam path should be understood to mean the totality of the measurement light beam 16 and the reflected-back measurement light 24.

Overall, the measurement beam path is confocal. In the case of the refractometer 10, “confocal” should be understood to mean that the measurement light source 14, to put it more prec3isely a confocal opening 26 provided as an opening in a pinhole stop 27, is imaged onto the retina 23, with the result that a light spot 28 that is as small as possible is produced on the retina, and light that impinges in the region of the light spot 28 is partly scattered by the retina 23, with the result that light energy emerges from the eye 12 as measurement light reflected back. A confocal opening 30 provided as an opening in a pinhole stop 31 is located in a plane conjugate to the retina 23 and to the opening 26 and at least partly transmits the reflected-back measurement light emerging from the eye 12. The intensity of the reflected-back measurement light 24 downstream of the confocal opening 30 is measured by the light detector 20. The opening 26 assigned to the measurement light source is focused onto the retina 23 by the optical unit 22, with the result that the light spot 28 on the retina 23 can be chosen to be as small as possible.

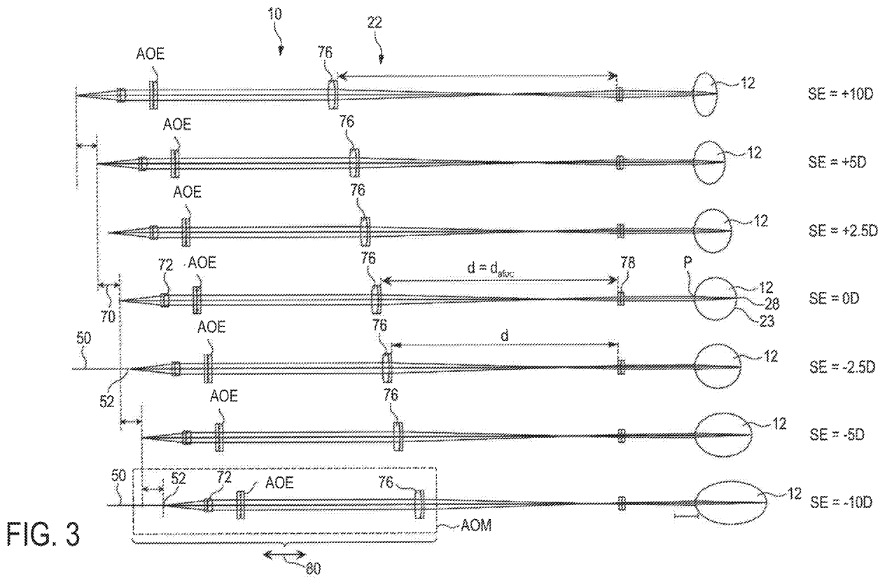

As shown in FIG. 1, the optical unit 22 further includes a lens group 32, by which the measurement light beam coming from the opening 26 is approximately collimated. The measurement light beam 16 thus collimated is guided via two beam splitters 34 and 36 into an optical arrangement AOM. The optical arrangement AOM contains, as will be described in even greater detail further below, one or more adaptive optical components AOE, as shown in FIG. 3. The optical arrangement AOM can generally contain optical components such as lenses, diffractive-optical elements, mirrors, beam splitters, etc., which can also be arranged in a displaceable manner.

In the context of the present description, a “lens group” is understood to mean either a single lens or, as shown in FIG. 1, an arrangement composed of a plurality of lenses, which can also have an air clearance between the individual lenses.

The optical arrangement AOM is settable by way of a control unit 38. The wavefront of the measurement light beam 16 incident in the optical arrangement AOM is altered depending on the setting of the optical arrangement AOM. The measurement light beam 16 entering the eye 12 generates a light spot 28 of greater or lesser size on the retina 23 of the eye 12. The impinging measurement light is scattered in the region of the light spot 28 on the retina 23. Part of the scattered light leaves the eye 12 as measurement light 24 reflected back. The measurement light 24 reflected back passes through the optical arrangement AOM in the opposite direction and is guided as an approximately collimated light beam via the beam splitter 36 and the beam splitter 34 to the confocal opening 30. A further lens group 33 focuses the measurement light 24 reflected back onto the opening 30.

The light detector 20 downstream of the opening 30 measures the intensity or the power of the reflected-back measurement light 24 that passes through the opening 30. The control unit 38 contains an arithmetic unit, for example, which can set the optical arrangement AOM by a suitable algorithm such that a maximum power is measured at the light detector 20. On the basis of the setting of the optical arrangement AOM for which the measured intensity of the measurement light 24 reflected back has a maximum, the measuring module 18 can then determine the refraction of the eye 12, in particular the spherical equivalent of the ametropia, the astigmatism and the axis position of the astigmatism, as will be described in greater detail later. The measuring module 18 and the control unit 38 can be embodied as a functional unit, wherein the function of the measuring module 18 can also be performed by the control unit 38, and vice versa.

It goes without saying that further optical components can be positioned between the eye 12 and the optical arrangement AOM.

The optionally provided beam splitter 36 can be followed by a display and/or an image sensor 40, wherein the display 40 and/or the image sensor 40 are/is arranged on an optical axis OA passing through the eye 12 and the optical arrangement AOM. A converging lens group 42 is arranged between the beam splitter 36 and the display and/or image sensor 40. The display 40 can be used to give the patient a stimulus for aligning the axis of the eye along the optical axis OA of the refractometer 10, or to give the patient a stimulus for accommodation. The display 40 can equally well also be used for subjective refraction measurement with the optical arrangement AOM being used as a phoropter. The image sensor 40 can serve to record an image of the front region of the eye 12 and to monitor whether the eye 12 is located in a suitable position relative to the refractometer 10.

FIG. 2 shows a modification of the confocal refractometer 10. For the confocal refractometer 10 shown in FIG. 2, the same reference signs as in FIG. 1 are used for elements which are identical, similar or comparable to elements of the refractometer 10 in FIG. 1.

In the case of the refractometer 10 in FIG. 2, the measurement light source 14 is connected to a first optical fiber 44 and the light detector 20 is connected to a second optical fiber 46. The first optical fiber 44 and the second optical fiber 46 are connected to a third optical fiber 50 via a fiber coupler 48, or merge into said third optical fiber. A free end 52 of the third optical fiber 50 forms an exit end for the measurement light beam 16 and an entrance end for the back-reflected measurement light 24. In this configuration, the confocality of the confocal refractometer 10 is achieved by virtue of the fact that the free end 52 of the optical fiber 50 simultaneously acts as a confocal opening and thus replaces the two confocal openings 30 and 26 of the confocal refractometer 10 shown in FIG. 1. In this configuration, moreover, the beam splitter 34 shown in FIG. 1 can be omitted, as is shown in FIG. 2. For the rest, reference can be made to the description of the refractometer 10 in FIG. 1.

The optical arrangement AOM of the refractometer 10 both shown in FIG. 1 and in FIG. 2 is configured to compensate for instances of spherical defective vision of the eye 12, e.g., by altering air clearances in the optical arrangement AOM or air clearances of the optical arrangement AOM with respect to other optical elements of the optical unit 22, with the result that different curvatures of the wavefront of the measurement light beam incident in the eye 12 can be produced at the pupil P of the eye 12. Furthermore, the optical arrangement AOM is configured to compensate for not only the spherical equivalent of the ametropia of the eye 12 but also an astigmatism for an arbitrary axis position of the astigmatism. For this purpose, the optical arrangement AOM includes one or more adaptive optical components AOE configured to compensate for the astigmatism in the wavefront of the measurement light by the setting of the adaptive optical component. The measuring module 18 can be configured to determine the astigmatism of the eye and the axis position of the astigmatism from a setting of the optical arrangement AOM and/or of the adaptive optical component(s) AOE for which the measured intensity of the back-reflected measurement light 24, as is measured by the light detector 20, has a maximum, as will also be described later.

Examples of adaptive optical components AOE which can be used to compensate for an astigmatism and the axis position thereof are Stokes lenses, Alvarez lenses, liquid-filled lenses, etc., will be described below.

Stokes lenses have two cylindrical lenses, which are rotatable relative to one another and of which one cylindrical lens has a positive refractive power C_{cyl} and a second cylindrical lens has an opposite negative refractive power −C_{cyl} of equal magnitude. If one of the cylindrical lenses is rotated by an angle θ and the other by the angle −θ, then the resulting cylindrical refractive power C_{SL} of the Stokes lens is given by C_{SL}=2C_{cyl}·sin(2θ). Consequently, a Stokes lens allows the production of a continuously adjustable cylindrical refractive power C_{SL}. It is possible to vary the axis position if both cylindrical lenses are rotated together. Stokes lenses of this type can be used in the optical arrangement AOM of the refractometer 10.

An Alvarez lens can compensate for not only an astigmatism but also a spherical equivalent of the ametropia. It includes two or more plates each having a surface contour, wherein the two surface contours are mutually complementary, and wherein the plates are translationally displaceable and/or rotatable relative to one another. The refractive power and/or the astigmatism of an Alvarez lens can be altered in a continuously variable manner by the plates being positioned accordingly with respect to one another.

In the case of liquid-filled lenses, the spherical and/or astigmatic refractive power is likewise variable. By way of example, it is possible to use two liquid-filled cylindrical lenses in the optical arrangement AOM, which lenses each have a variable refractive power and are crossed with respect to one another by an angle of not equal to 0°, e.g., of 45°. The cylindrical lenses are positionally fixed with respect to one another, wherein the astigmatism and the axis position of this combination of cylindrical lenses can be varied in a continuously adjustable manner by the astigmatic refractive powers of the two individual cylindrical lenses being set in a suitable manner. In this configuration, the adaptive optical component of the optical arrangement AOM is variable with regard to its astigmatic power including axis position, without the adaptive optical component having to be moved mechanically.

Typically, the adaptive optical component is settable into a neutral setting independently of its specific configuration, the adaptive optical component having no astigmatic power in said neutral setting. Such a neutral setting is provided in the case of the above-described examples of a Stokes lens, an Alvarez plate or liquid-filled lenses.

The confocal refractometers 10 shown in FIGS. 1 and 2 can be used to determine the spherical equivalent SE, the astigmatism C_{eye} and the axis position φ of the astigmatism of the examined eye 12. The spherical equivalent SE is a statistic for the ametropia. Both the spherical equivalent SE and the astigmatism C_{eye} are usually specified in dioptres (D). Usually, eyes with the spherical equivalent of SE<0 D are referred to as nearsighted or myopic, while eyes with SE>0 D are referred to as farsighted or hyperopic. Patients' eyes with SE≈0 D are referred to as having spherically perfect vision or as emmetropic. The astigmatism C_{eye} specifies the difference between the refractive powers of the eye 12 in two mutually perpendicular principal meridians. The axis position φ specifies the position of these principal meridians, represents an angle and is specified in the unit of degrees (°).

The following convention is used in the present description. The astigmatism C_{eye} is always positive and satisfies C_{eye}>0 D. In the two principal meridians, the defective vision of the patient's eye is described by SE±(1/2)C. The axis position φ describes the position of that principal meridian with the defective vision SE+(1/2) C. For known values SE, C_{eye}, and φ, it is possible to produce a spectacle lens that corrects instances of defective vision of the patient's eye. It goes without saying that it is also possible to use other conventions for describing defective vision, but they can always be converted into the convention indicated above.

An exemplary more detailed configuration of an optical arrangement AOM is described below with reference to FIG. 3. FIG. 3 shows an optical unit 22 having an optical arrangement AOM, wherein the optical unit 22 can be used in particular in the refractometer 10 in accordance with FIG. 2. FIG. 3 shows only the confocal optical unit 22 of the refractometer, while the other components such as measurement light source, light detector, measuring module and control unit have been omitted for reasons of clarity. The reference signs which are used in FIG. 3 and are identical to reference signs in FIG. 2 refer to the elements described with reference to FIG. 2.

In FIG. 3, ellipses at the right-hand edge of the figure show schematic patients' eyes having spherical equivalents of from SE=−10 D (bottom) to SE=+10 D (top).

The optical arrangement AOM includes a first lens group 72, an adaptive optical component AOE and a further lens group 76. The end 52 of the optical fiber 50 is located near a focal plane of the first lens group 72, which collimates the measurement light beam emerging from the end 52 of the optical fiber 50. The focal length of the lens group 72 is 40 mm, for example. The adaptive optical component AOE can be embodied, e.g., as a Stokes lens including cylindrical lenses, e.g., having a cylindrical refractive power C_{cyl}=+1 D. The adaptive optical component AOE is arranged near a focal plane of the second lens group 76. The second lens group 76 has, e.g., a focal length f₇₆ of 150 mm. A third lens group 78 is arranged such that the pupil P of the eye 12 is located near the focal plane of the third lens group 78. The third lens group 78 has, e.g., a focal length f₇₈ of 60 mm. The lens groups 76 and 78 form, e.g., a Kepler telescope.

The optical arrangement AOM is suitable for compensating for spherical defective vision, astigmatism and the axis position thereof. The optical arrangement AOM is movable as a whole along the optical axis in accordance with a double-headed arrow 80, e.g., by virtue of the optical arrangement AOM being mounted on a slide, with the result that the distance between the optical arrangement AOM and the third lens group 78 can be varied. The respectively set position of the optical arrangement AOM can be specified by a distance d between the two lens groups 76 and 78.

The end 52 of the optical fiber 50, the lens group 72, the adaptive optical component AOE and the lens group 76 are positionally fixed with respect to one another in the direction of the optical axis (arrow 80). In the exemplary case of the configuration of the adaptive optical component AOE as a Stokes lens, the two associated cylindrical lenses are rotatable relative to one another about the optical axis in order to vary the cylinder power of the Stokes lens, and rotatable jointly in order to vary the axis position of the Stokes lens.

The measurement light beam emerging from the end 52 of the optical fiber 50 is collimated by the lens group 72, passes through the adaptive optical component AOE and also the lens groups 76 and 78 and enters the patient's eye 12 through the pupil P, where it produces a light spot 28 on the retina 23. The light scattered at the light spot 28 partly leaves the eye 12 again as reflected-back measurement light, passes through the optical unit 22 in the opposite order and is focused by the first lens group 72 onto the end 52 of the optical fiber 50, where it is partly coupled into the optical fiber 50, and passes through the fiber coupler 48 (see FIG. 2). The intensity of the reflected-back measurement light reaching the light detector 20 is measured by the light detector 20 (see FIG. 2). During the measurement of the intensity of the reflected-back measurement light, the optical arrangement AOM including the adaptive optical component AOE can be set until the intensity measured at the light detector 20 becomes maximal, wherein a maximal intensity of the reflected-back measurement light means that the spherical equivalent of the ametropia and the astigmatism and also the axis position thereof for the patient's eye are compensated for.

A description is given below of exemplary embodiments of measuring methods for determining an astigmatism of a patient's eye with the aid of a confocal refractometer, e.g., the refractometer 10 in FIG. 2 having the optical unit 22 in FIG. 3.

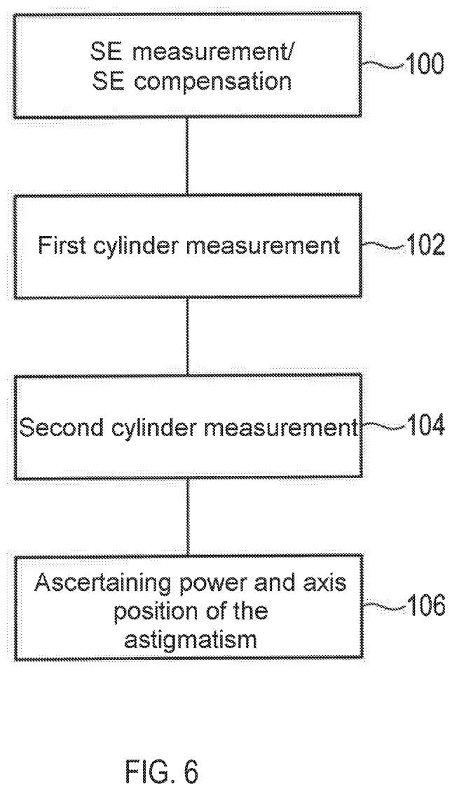

FIG. 6 shows a flowchart of a first exemplary embodiment of a measuring method of this type.

As already described above, in the measuring method, a measurement light beam 16 is directed onto the eye 12, with the result that a light spot is produced on the retina 23 of the eye 12. An intensity of measurement light 24 reflected back from the retina 23 is measured.

A step 100 in accordance with FIG. 6 involves firstly performing a measurement and/or compensation of the spherical equivalent of the ametropia. In step 102, a first cylinder measurement is carried out. In a step 104, a second cylinder measurement is carried out. In a step 106, the power and axis position of the astigmatism are determined from the cylinder measurement values resulting from the first cylinder measurement and the second cylinder measurement.

Firstly, a description is given of the measurement and/or compensation of the spherical equivalent SE of the ametropia of the eye 12 by way of example with reference to FIG. 3. While the intensity of the measurement light reflected back from the retina 23 is measured by the light detector 20 (see FIG. 2), the optical arrangement AOM is displaced in accordance with the arrow 80 until a maximum intensity is measured at the light detector 20. In this case, the adaptive optical component AOE is typically set in its neutral position, with the result that it does not exhibit any astigmatic power. In a position of the optical arrangement AOM in which the focal points of the lens groups 76 and 78 coincide, the associated distance d between the lens groups 76 and 78 is referred to hereinafter as d_{afoc}. For the distance d_{afoc} it holds true approximately that d_{afoc}≈f₇₆+f₇₈. In the abovementioned example for the focal lengths f₇₆ and f₇₈ of 150 mm and 60 mm a value of approximately 210 mm results for the distance d_{afoc}. A more accurate determination of d_{afoc} for given lens groups 76 and 78 is possible both computationally and experimentally.

The optical unit 22 in FIG. 3 can be configured in particular such that the adaptive optical component AOE is arranged approximately in the focal plane of the lens group 76, and the pupil P of the eye 12 is located near the focal plane of the lens group 78. In this arrangement, the adaptive and optical component AOE and the pupil P are located in mutually conjugate planes.

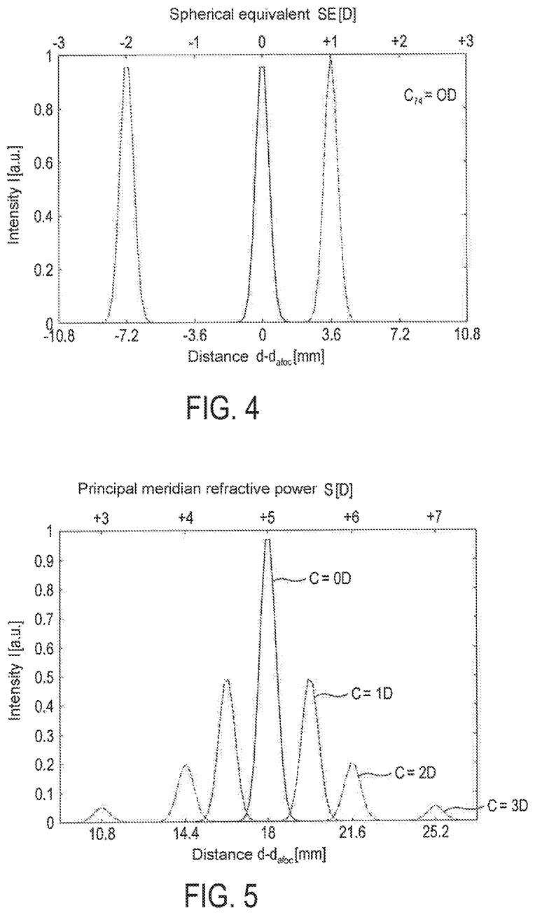

FIG. 4 shows exemplary measurements of the intensity I for various patients' eyes free of astigmatism and having a different spherical equivalent SE. The difference between the set distance d and the distance d_{afoc} is plotted on the abscissa of the diagram in FIG. 4, and the associated intensity on the ordinate.

From the distance d for which the measured intensity I assumes its maximum, in this case the spherical equivalent SE can be determined computationally by the equation

$$d - {d}_{afoc} = SE \cdot {f}_{78}^{2} = \frac{S E}{D} \cdot 36 mm ,$$

when the exemplary value mentioned above for the focal length f₇₈ is used.

In step 100, by displacing the optical arrangement AOM in FIG. 3, the focus position of the measurement beam path is thus varied until the measured intensity of the measurement light 24 reflected back from the retina 23 is maximal. The spherical equivalent of the ametropia can then be determined from the displacement distance d−d_{afoc}.

After the measurement of the spherical equivalent of the ametropia has been carried out and in order that the spherical equivalent of the ametropia is compensated for in the measurement beam path, the first cylinder measurement 102 is carried out. During the first cylinder measurement, the measurement light beam 16 is still directed onto the eye 12, and the intensity of the measurement light 24 reflected back from the retina 23 is still measured.

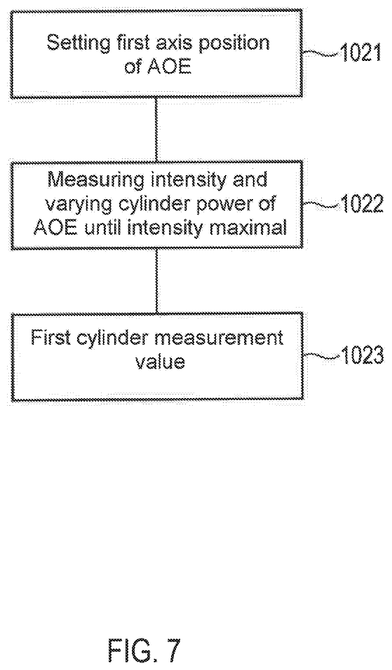

FIG. 7 shows a flow diagram illustrating individual steps of the first cylinder measurement 102. In a step 1021, a fixed cylindrical axis position of the adaptive optical component AOE is set. In a step 1022, the intensity of the measurement light 24 reflected back from the retina 23 is measured. In this case, the cylinder power of the adaptive optical component AOE is varied until the measured intensity becomes maximal. A first cylinder measurement value is obtained from this in a step 1023, said first cylinder measurement value resulting from the cylinder power of the component AOE for which the measured intensity is maximal.

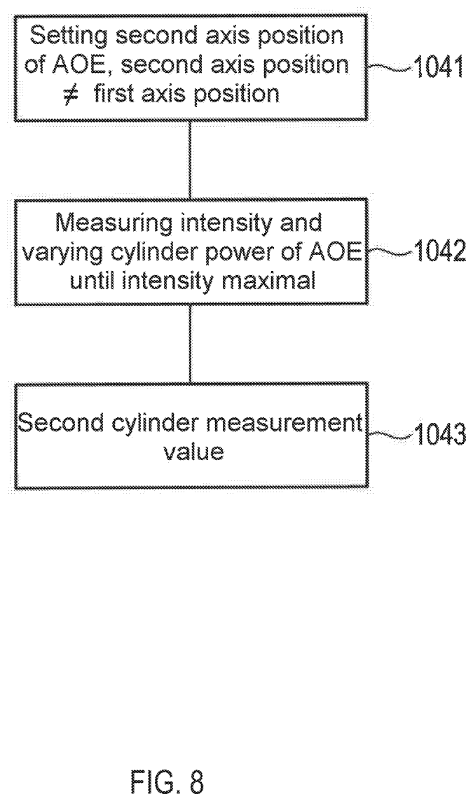

After the first cylinder measurement 102, the second cylinder measurement 104 is carried out. FIG. 8 shows a flow diagram of individual steps of the second cylinder measurement 104. In a step 1041, a second fixed axis position is set at the adaptive optical component AOE, said second fixed axis position being different than the first axis position in the first cylinder measurement 102. In a step 1042, once again the intensity of the measurement light 24 reflected back from the retina 23 is measured. In this case, the cylinder power of the adaptive optical component AOE is varied until the measured intensity is maximal again. In a step 1043, a second cylinder measurement value b is obtained, which then corresponds to the cylinder power which is set at the component and for which the measured intensity is once again maximal.

In the step 106 in FIG. 6, the power of the astigmatism C_{eye} and the axis position φ thereof are then determined by calculation from the first measurement value a, the second measurement value b, the first axis position φ1 and the second axis position φ2=φ1+Δβ of the adaptive optical component AOE. A description is given below of how the power of the astigmatism C_{eye} and the axis position φ thereof are calculated.

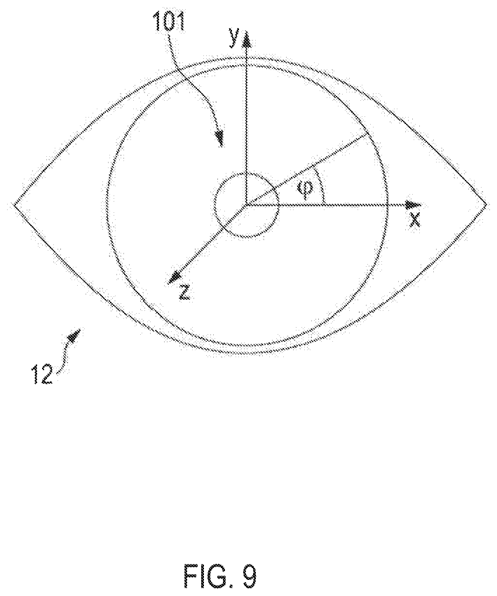

If the spherical equivalent of the ametropia is compensated for in the measurement beam path, there is located on the retina 23 either a focus point if the patient's eye 12 is not afflicted with astigmatism, or a “circle of least confusion” if the patient's eye is afflicted with astigmatism. A change in the power of the cylinder of the adaptive optical component AOE does not influence the spherical equivalent. As a result of the imaging of the adaptive optical component AOE into the vicinity of the pupil P of the patient's eye 12, the astigmatism of the adaptive optical component AOE and the astigmatism of the eye are superimposed. The astigmatism of the adaptive optical component and the astigmatism of the eye can be described by the Zernike polynomials since the slightly elliptical pupil P of the patient can be assumed to be approximately round. FIG. 9 shows a coordinate system 101 in relation to the patient's eye 12, wherein FIG. 9 shows a front view, such as is seen by a physician, both for the right and for the left eye. The z-axis points out of the plane of the drawing in FIG. 9. The x-axis is the horizontal axis of the eye. Both the angle θ of the Zernike polynomials and the angle φ of the axis position of the astigmatism are measured with respect to the x-axis. The radius R is measured in the xy-plane with respect to the coordinate origin.

The vertical astigmatism is described by the Zernike polynomial Z₅=R² cos(2θ) and the 45° astigmatism is described by the Zernike polynomial Z₆=R² sin(2θ). The power of the astigmatism is described by an additional coefficient in the respective polynomial. Any arbitrary astigmatism Ast can be described as a superimposition of the two polynomials Z₅ and Z₆:

Ast=c₅·Z₅+c₆·Z₆ (1)

The superimposition of the two polynomials can also be combined into one polynomial:

Ast=c_{Ast}·R² sin(2(θ+α)) (2)

With the aid of the addition theorem sin(x+y)=sin(x)cos(y)+cos(x)sin(y), it is possible to rearrange the sin term:

Ast=c_{Ast}·R²(sin(2θ)cos(2(α))+cos(2θ)sin(2α)) Ast=c_{Ast}·cos(2α)·Z₆+c_{Ast}·sin(2α)·Z₅=c₆·Z₆+c₅·Z₅ (3)

For the coefficients c₅ and c₆ it additionally holds true that:

c₅²+c₆²=c _{Ast}² cos²(2α)+c_{Ast}² sin²(2α)=c_{Ast}²(cos²(2α)+sin²(2α))=c_{Ast}² (4)

The coefficient c_{Ast} is thus calculated with:

c_{Ast}=\sqrt{c₅²+c₆²} (5)

If the power of two cylindrical lenses C₁ and C₂ is superimposed, this superimposition can likewise be described by an addition of the Zernike polynomials. If both cylindrical lenses are oriented identically and the axes are in each case oriented in such a way that both cylindrical lenses produce a 45° astigmatism, then the resulting astigmatism can be described as follows:

Ast=C₁ sin(2θ)+C₂ sin(2θ) (6)

If one cylindrical lens is rotated by the angle +α in the xy-plane, and the other cylindrical lens by the angle −α, the following results:

$$\begin{matrix}Ast = {C}_{1} sin ( 2 ( θ + α ) ) + {C}_{2} sin ( 2 ( θ - α ) ) \\ = {C}_{1} [ sin ( 2 θ ) cos ( 2 α ) + cos ( 2 θ ) sin ( 2 α ) ] + \\ {C}_{2} [ sin ( 2 θ ) cos ( - 2 α ) + cos ( 2 θ ) sin ( 2 - α ) ] \\ = {C}_{1} [ sin ( 2 θ ) cos ( 2 α ) + cos ( 2 θ ) sin ( 2 α ) ] + \\ {C}_{2} [ sin ( 2 θ ) cos ( 2 α ) - cos ( 2 θ ) sin ( 2 α ) ] \\ = [ ( {C}_{1} + {C}_{2} ) cos ( 2 α ) ] \cdot sin ( 2 θ ) + \\ [ ( {C}_{1} - {C}_{2} ) sin ( 2 α ) ] \cdot cos ( 2 θ )\end{matrix} \qquad ( 7 )$$

This in turn corresponds to an addition of the polynomials Z₅ and Z₆, with coefficients that are dependent on the angle α of rotation. In order to deduce the power of the astigmatism, the coefficients of Z₅ and Z₆ can be summed as described above to form a coefficient A(α):

$$\begin{matrix}{A}^{2} ( α ) = {[ ( {C}_{1} + {C}_{2} ) cos ( 2 α ) ]}^{2} + {[ ( {C}_{1} - {C}_{2} ) sin ( 2 α ) ]}^{2} \\ = ( {C}_{1}^{2} + 2 {C}_{1} {C}_{2} + {C}_{2}^{2} ) {cos}^{2} ( 2 α ) + \\ ( {C}_{1}^{2} - 2 {C}_{1} {C}_{2} + {C}_{2}^{2} ) {sin}^{2} ( 2 α ) \\ = {C}_{1}^{2} + 2 {C}_{1} {C}_{2} ( 2 α ) - {sin}^{2} ( 2 α ) + {C}_{2}^{2}\end{matrix} \qquad ( 8 )$$

This yields with cos²x−sin²x−cos(2x), the general case for the superimposition of two cylindrical lenses:

A(α)=\sqrt{C₁²+2C₁C₂ cos(4α)+C₂²} (9)

The principal meridians or axes of the two cylindrical lenses here form the angle 2α.

The Zernike coefficients can be converted to obtain the customary spherocylindrical representation in diopters. In this case, there is a linear relationship between the refractive power and the associated Zernike coefficient (see Wesemann, W. “Mathematical note: What relationship is there between the normal spherocylindrical notation of corrective lenses and the Zernike polynomials?” in DOZ, issue 3/2005, pages 40-44). The same formula can thus be applied to the notation with refractive powers in diopters.

As a result of the imaging of the adaptive optical component AOE into the vicinity of the pupil of the eye, the system including adaptive optical component AOE and eye can likewise be regarded as a superimposition of two cylindrical lenses, where

C₁=C_{AOE} and C₂=C_{eye}=C

The power of the cylinder of the adaptive optical component AOE is settable in a variable manner, as is the angle 2α. However, since the axis position of the cylinder of the eye is initially unknown, 2α is also unknown. The astigmatism that arises from the superimposition of the two cylinders (AOE and eye) is intended to become as small as possible in order to measure a high intensity of the measurement light 24 reflected back from the retina 23, that is to say:

A(C_{AOE},α)=min (10)

The zero of the first derivative of A with respect to CAOE is thus calculated:

$$\begin{matrix}\frac{\partial A ( {C}_{A O E} , α )}{\partial {C}_{A O E}} = 0 & ( 11 ) \\ \frac{2 {C}_{A O E} + 2 C \cdot cos ( 4 α )}{2 \sqrt{{C}_{A O E}^{2} + 2 {C}_{A O E} C \cdot cos ( 4 α ) + {C}^{2}}} = 0 & ( 12 ) \\ {C}_{A 0 E} = - C \cdot cos ( 4 α ) & ( 13 )\end{matrix}$$

where C_{eye}=C.

In the case of the measuring method in accordance with the present exemplary embodiment, with a compensated spherical equivalent SE and an arbitrary, but known, first axis position of the adaptive optical component AOE, with the result that the axis of the adaptive optical component AOE and the axis of the cylinder of the eye form the angle β₁, the power of the astigmatism C_{AOE} is varied until a maximal intensity of the measurement light 24 reflected back from the retina 23 is measured (step 102).

The measurement value a is thus obtained:

α=−C·cos(2β₁) (14)

The first measurement value a is that cylinder power C_{AOE} set at the adaptive optical component for which the measured intensity is maximal, and C=C_{eye}.

The first axis position of the adaptive optical component AOE is then altered by an arbitrary, but known, angle Δβ into a second axis position. The angle between the axis positions of the cylinder of the eye and of the cylinder of the adaptive optical component AOE is then β₂=β₁+Δβ. The power of the astigmatism C_{AOE} is once again varied until a maximal intensity of the measurement light 24 reflected back from the retina 23 is measured (step 104). The second measurement value b results:

b=−C·cos(2(β₁+Δβ)) (15)

Both equations (14) and (15) can be solved with respect to (−C) and then equated:

$$\frac{a}{cos ( 2 {β}_{1} )} = \frac{b}{cos ( 2 ( {β}_{1} + Δ β ) )} \qquad ( 16 )$$

After application of the addition theorem:

cos(x+y)=cos x cos y −sin x sin y (17)

and rearrangement, the following is obtained:

cos(2β₁)·[α·cos(2Δβ)−b]=α·sin(2β₁) sin(2Δβ) (18)

The tangent of the angle 2β₁ can thus be calculated:

$$tan ( 2 {β}_{1} ) = \frac{a \cdot cos ( 2 Δβ ) - b}{a \cdot sin ( 2 Δ β )} \qquad ( 19 )$$

The sought power of the astigmatism C=C_{eye} of the eye is then:

$$C = - \frac{a}{cos ( 2 {β}_{1} )} \qquad ( 20 )$$

For the angle β₁ it holds true that:

β₁=φ−φ₁ (21)

wherein φ is the axis position of the astigmatism of the eye and φ₁ is the axis position of the adaptive optical component AOE in the first measurement 102.

For simplification, it is appropriate to choose φ₁=0° and Δβ=45°. This results in:

C=−\sqrt{α²+b²} (22)

and

$$tan ( 2 {β}_{1} ) = \frac{- b}{a} \qquad ( 23 )$$

The following then results for the axis position φ of the astigmatism of the eye:

$$ϕ = \frac{arctan ( \frac{- b}{a} )}{2} \qquad ( 24 )$$

The spherical equivalent SE of the ametropia of the patient's eye 12 is known from step 100 of the measurement. The first cylinder measurement 102 and the second cylinder measurement 104 yield the measurement values a and b, and the latter and the known first and second axis positions of the adaptive optical component AOE in the measurements 102 and 104 yield the axis position φ of the patient's eye 12 with the formulae (19) and (21) or with the formula (24) (for Δβ=45° and φ₁=0°) and also the power of the astigmatism C_{eye} of the patient's eye with the formula (20) or (22) (for Δβ=45° and φ₁=0°.

The difference Δβ between the first and second axis positions of the adaptive optical component AOE (74) can be in an angular range of 30° to 45°.

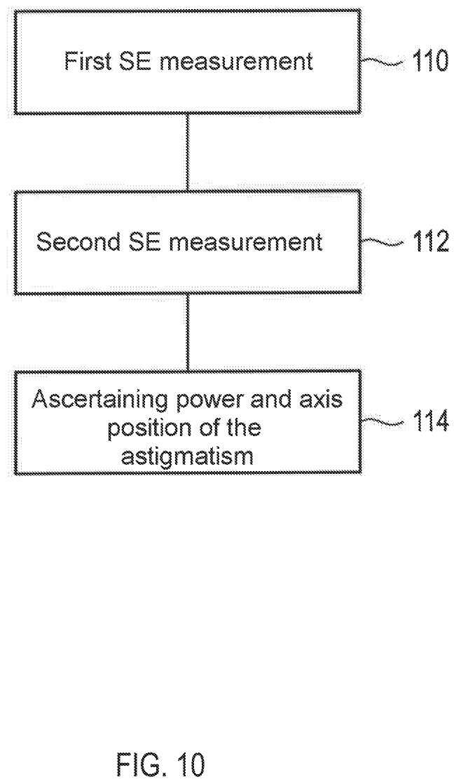

A further exemplary embodiment of a measuring method for determining an astigmatism of an eye with the aid of a confocal refractometer is described with reference to FIG. 10 to 12. For example, as in the previous exemplary embodiment, the confocal refractometer can be the refractometer 10 in FIG. 2 having the optical unit 22 in FIG. 3. In the measuring method in accordance with the present exemplary embodiment, a measurement light beam 16 is directed onto the eye 12, with the result that a light spot is produced on the retina 23 of the eye. An intensity of measurement light 24 reflected back from the retina 23 is measured. In accordance with a step 110 a first measurement of the spherical equivalent of the ametropia is carried out. In a step 112, a second measurement of the spherical equivalent of the ametropia of the eye 12 is carried out. A step 114 involves determining or calculating the power and axis position of the astigmatism of the patient's eye from the first and second measurements of the spherical equivalent of the ametropia.

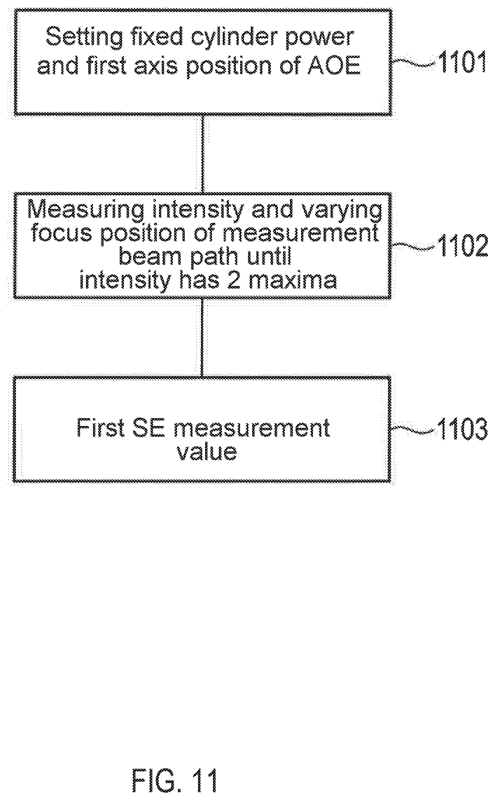

FIG. 11 shows individual steps of the first measurement of the spherical equivalent SE. In the case of the first SE measurement, in a step 1101, a fixed cylinder power, which can be not equal to 0 D, and a fixed first cylindrical axis position are set during the first SE measurement at the adaptive optical component. In a step 1102, the intensity is measured and in this case the focus position of the optical arrangement AOM (FIG. 3) is varied until the measured intensity of the measurement light 24 reflected back from the retina 23 is maximal twice. In a step 1103, a first SE measurement value is obtained as the result of the first SE measurement.

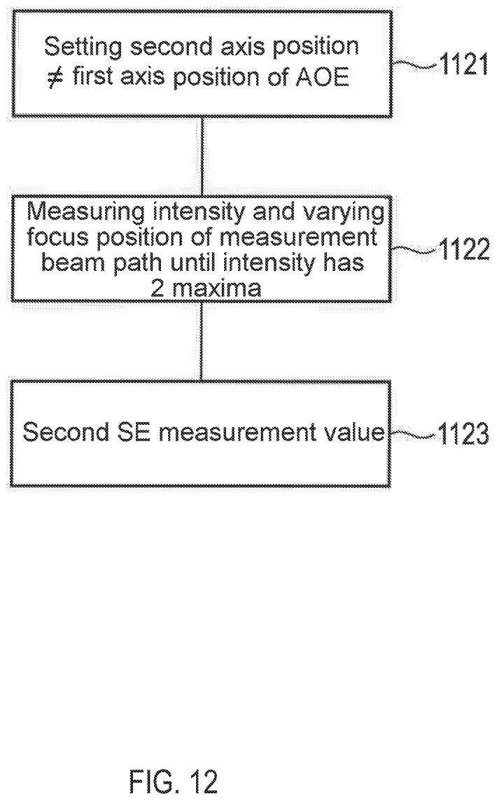

FIG. 12 shows individual steps of the second SE measurement 112. A step 1121 involves setting a second fixed axis position at the adaptive optical component 74, said second fixed axis position being different than the first fixed axis position of the adaptive optical component 74. The cylinder power of the adaptive optical component AOE set in step 1101 is maintained. In a step 1122, the intensity of the measurement light 24 reflected back from the retina 23 is measured and in this case the focus position of the optical arrangement AOM is varied until the intensity is once again maximal twice. A second SE measurement value is obtained as the result in step 1123.

The occurrence of two intensity maxima is explained below with reference to FIG. 5. FIG. 5 shows by way of example the measured intensity I when eyes are characterized by astigmatisms C having different powers for one respectively identical spherical equivalent SE=+5 D. While the measurement curve has only one maximum in the astigmatism-free case where C=0 D, two maxima occur in the case of C>0 D. These two maxima of the intensity I arise if, in the example of the refractometer 10 in FIG. 2 having the optical unit 22 in FIG. 3, the end 52 of the optical fiber 50 is imaged onto the retina 23 in a focused manner in one of the two principal meridians. Specifically, since an eye with astigmatism in both principal meridians has a different refractive power, there are two distances d in FIG. 3 for which the intensity I becomes maximal, specifically d₁ and d₂ with d₁<d₂. If the eye 12 has only a small astigmatism, it may happen that both maxima merge together and only one widened maximum is resolvable. In this case, the two distances d₁ and d₂ are identical. If such a case occurs, it is advantageous to alter the cylinder power of the adaptive optical component AOE in the first SE measurement. The distance Δ=d₂−d₁ between the intensity maxima is approximately related to the astigmatism of the eye (for the abovementioned exemplary value of the focal length f78) by way of the equation Δ=C·f²₇₈=(C/D)·3.6 mm. The spherical equivalent SE of an eye afflicted with astigmatism results from d₁ and d₂ in accordance with the relationship (d₁+d₂)/2−d_{afoc}=SEZ·f²₇₈=(SE/D)·3.6 mm.

From the displacement distance of the optical arrangement AOM in FIG. 3 and the resultant distance between the two intensity maxima as a function of the displacement distance, it is thus possible to determine the first SE measurement value and the second SE measurement value in diopters. The first SE measurement value is designated hereinafter by A1 and the second SE measurement value is designated hereinafter by A₂.

The way in which the power of the astigmatism of the patient's eye and the axis position thereof can be determined from the two SE measurement values A₁ and A₂ is described below.

In the case of eyes having astigmatism, as described above, two maxima usually occur during the measurement of the spherical equivalent of the ametropia. If an astigmatism is set at the adaptive optical component AOE, it is imaged onto the eye 12. As a result, two maxima likewise occur during the measurement of the spherical equivalent. If two cylindrical lenses having cylinder powers C₁ and C₂, in this case the cylindrical lens given by the adaptive optical component AOE and the cylindrical lens given by the eye 12, are superimposed with one another, and the principal meridians of the two cylindrical lenses C₁ and C₂ together form the angle β, the resulting cylindrical refractive power is:

A=\sqrt{C₁²+2C₁C₂ cos(2β)+C₂²} (25)

Here C₁=C_{AOE} and C₂=C_{eye}=C.

The two measurement values A₁ and A₂ are:

A₁=\sqrt{C_{AOE}²+2C_{AOE}C cos(2β₁)+C²} (26)

A₂=\sqrt{C_{AOE}²+2C_{AOE}C cos(2β₂)+C²} (27)

where β₂=β₁+δ, wherein β₁ is the angle between the principal meridians of the two cylindrical lenses C_{AOE} and C=C_{eye} in the first SE measurement and δ is the difference between the axis positions of the adaptive optical component in the first and second SE measurements. C is the cylinder power or the power of the astigmatism C_{eye} of the eye 12. If δ=90° is advantageously chosen, the following results from the two equations above:

$$C = \sqrt{{A}_{1}^{2} - {C}_{A O E}^{2} - \frac{{A}_{1}^{2} - {A}_{2}^{2}}{2}} \qquad ( 28 )$$

In order to determine the axis position φ of the astigmatism of the eye 12, firstly the angle β₁ is calculated:

$$\begin{matrix}cos ( 2 {β}_{1} ) = \frac{{A}_{1}^{2} - {A}_{2}^{2}}{4 {C}_{A O E} C} & ( 29 ) \\ {β}_{1} = \frac{arccos ( \frac{{A}_{1}^{2} - {A}_{2}^{2}}{4 {C}_{AOE} C} )}{2} & ( 30 )\end{matrix}$$

The angle β₁ is the angle between the principal meridians of the cylinder of the eye and of the cylinder of the adaptive optical component AOE. For a known axis position φ₁ of the adaptive optical component AOE, the axis position φ of the eye 12 can thus be calculated:

φ=φ₁+β₁ (31)

Since an astigmatism of the adaptive optical component AOE does not influence the spherical equivalent of the ametropia of the patient's eye, with the first SE measurement and the second SE measurement, from the position of the intensity maxima, as described above, it is possible to determine the sought spherical equivalent of the patient's eye (see above). With the SE measurement values A₁ and A₂ and also the known value of the set cylinder power C_{AOE}, the power of the astigmatism C_{eye} of the patient's eye can be calculated using formula (28). With the calculated value C_{eye} and the measurement values A₁ and A₂ and also the set known cylinder power C_{AOE} of the adaptive optical component, the axis position φ of the astigmatism of the eye 12 can in turn be determined using formulae (30) and (31).

It is understood that the foregoing description is that of the preferred embodiments of the invention and that various changes and modifications may be made thereto without departing from the spirit and scope of the invention as defined in the appended claims.

What is claimed is:

1. A measuring method for determining an astigmatism of an eye with a confocal refractometer providing a measurement beam path, an optical arrangement having an adaptive optical component with a variable cylinder power and a variable cylindrical axis position, the optical arrangement being arranged in the measurement beam path to compensate for the astigmatism of the eye in a wavefront of the measurement beam path, the method comprising:
directing a measurement light beam onto the eye such that a light spot is produced on a retina of the eye;
measuring an intensity of measurement light reflected back from the retina, in a first measurement a cylinder power of the adaptive optical component for a first fixed cylindrical axis position of the adaptive optical component being varied until a measured intensity becomes maximal to obtain a first measurement value, and in a second measurement the cylinder power of the adaptive optical component for a second fixed cylindrical axis position of the adaptive optical component being varied until the measured intensity is maximal to obtain a second measurement value, the second fixed cylindrical axis position being different from the first fixed cylindrical axis position; and
determining a power and axis position of the astigmatism of the eye at least from the first and second measurement values.

2. The measuring method as claimed in claim 1, wherein:
the optical arrangement is further configured to vary a focus position of the measurement beam path to compensate for a spherical equivalent of an ametropia of the eye in the wavefront of the measurement beam path, and
before the first measurement with the optical arrangement, the focus position of the measurement beam path is varied until the measured intensity of measurement light reflected back from the retina is maximal.

3. The measuring method as claimed in claim 2, wherein the cylinder power of the adaptive optical component is set to zero during a variation of the focus position.

4. The measuring method as claimed in claim 2, further comprising:
determining the spherical equivalent of the ametropia from the setting of the optical arrangement in which the spherical equivalent of the ametropia of the eye is compensated for.

5. The measuring method as claimed in claim 1, wherein:
the first measurement value and the second measurement value are linked with the power of the astigmatism of the eye and the axis position φ of the astigmatism of the eye by:
a=−C_{eye}·cos(2β₁),
b=−C_{eye}·cos(2β₂),
β₁=φ−φ₁,
β₂=φ−φ₂,
φ₁ is the axis position of the adaptive optical component in the first measurement,
φ₂ is the axis position of the adaptive optical component in the second measurement, and
C_{eye} is the astigmatism of the eye.

6. The measuring method as claimed in claim 1, wherein the axis position φ of the astigmatism of the eye is determined from the first measurement value, the second measurement value, the first axis position φ₁ of the adaptive optical component and a difference Δβ between the first and second axis positions of the adaptive optical component in accordance with:
φ=(1/2)·arctan((a·cos(2Δβ)−b)/(a·sin(2Δβ)))+φ₁.

7. The measuring method as claimed in claim 1, wherein a second axis position of the adaptive optical component differs from a first axis position of the adaptive optical component by an angle in a range of 30° to 45°.

8. The measuring method as claimed in claim 1, wherein a first axis position of the adaptive optical component is set to 0° with respect to a horizontal axis of the eye and a second axis position is altered by 45° relative to the first axis position.

9. The measuring method as claimed in claim 8, wherein the power of the astigmatism of the eye is determined from the first measurement value and the second measurement value in accordance with:
C_{eye}=−(a²+b²)^{1/2},
a is the first measurement value,
b is the second measurement value, and
C_{eye} is the astigmatism of the eye.

10. The measuring method as claimed in claim 8, wherein:
the axis position φ of the astigmatism of the eye is determined from the first measurement value and the second measurement value in accordance with:
φ=1/2 arctan (−b/a),
a is the first measurement value, and
b is the second measurement value.

11. A measuring method for determining an astigmatism of an eye with a confocal refractometer providing a measurement beam path, an optical arrangement having an adaptive optical component with a variable cylinder power and a variable cylindrical axis position, the optical arrangement being arranged in the measurement beam path to compensate for the astigmatism of the eye in a wavefront of the measurement beam path, and the optical arrangement being configured to vary a focus position of the measurement beam path to compensate for a spherical equivalent of an ametropia of the eye in the wavefront of the measurement beam path, the method comprising:
directing a measurement light beam onto the eye such that a light spot is produced on a retina of the eye;
measuring an intensity of measurement light reflected back from the retina, in a first measurement the focus position of the measurement beam path for a fixed first cylinder power of the adaptive optical component and a fixed axis position of the adaptive optical component being varied until a measured intensity is maximal to obtain a first measurement value, and in a second measurement the focus position of the measurement beam path for a first cylinder power and a second axis position of the adaptive optical component being varied until the measured intensity is maximal to obtain a second measurement value, the second axis position being different from a first axis position; and
determining a power of the astigmatism of the eye and an axis position thereof at least from the first and second measurement values.

12. The measuring method as claimed in claim 11, wherein:
in the first and second measurements, the focus position is varied until the measured intensity of measurement light reflected back has respectively a first and a second intensity maximum, and
the first and second measurement values are respectively determined from the setting of the optical arrangement which is associated with the first and second intensity maxima.

13. The measuring method as claimed in claim 11, wherein, when in the first measurement the intensity of measurement light reflected back, upon varying the focus position of the measurement beam path, has only one maximum, a cylinder power of the adaptive optical component is altered, resulting in two maxima of the intensity of measurement light reflected back to occur.

14. The measuring method as claimed in claim 11, wherein:
the first measurement value and the second measurement value are linked with the power of the astigmatism of the eye by:
A_{1,2}=(C_{AOE}²+2C_{AOE}C_{eye}·cos(2β_{1,2})+C_{eye}²)^{1/2},
C_{AOE} is a set first cylinder power of the adaptive optical component,
β₁ is an angle between principal meridians of cylinders of the adaptive optical component and of the eye in the first measurement,
β₂ is the angle between the principal meridians of the cylinders of the adaptive optical component and of the eye in the second measurement, and
C_{eye} is the astigmatism of the eye.

15. The measuring method as claimed in claim 14, wherein the axis position of the adaptive optical component in the second measurement is rotated by 90° relative to the axis position of the adaptive optical component in the first measurement.

16. The measuring method as claimed in claim 15, wherein:
the power of the astigmatism of the eye is determined from the first and second measurement values and the set first cylinder power of the adaptive optical component in accordance with:
C_{eye}=(A₁ ²−C_{AOE}²−1/2(A₁²−A₂²))^{1/2},
A₁ is the first measurement value,
A₂ is the second measurement value,
C_{eye} is the astigmatism of the eye, and
C_{AOE} is the set first cylinder power of the adaptive optical component.

17. The measuring method as claimed in any of claim 14, wherein the angle β₁ between the principal meridians of the cylinders of the adaptive optical component and of the eye in the first measurement is given by:
β₁=1/2 arccos((A₁²−A₂²)/(4C_{AOE} C_{eye})).

18. The measuring method as claimed in claim 14, wherein:
the axis position φ of the astigmatism of the eye is determined as φ=φ₁+β₁, and
φ₁ is the axis position of the adaptive optical component in the first measurement.
